(19) United States
(12) Patent Application Publication
(10) Pub. No.: US 2010/0064421 A1
(43) Pub. Date: Mar. 18, 2010

(54) Protective Goggle Assembly

(76) Inventor: Tzu-Feng Wang-Lee, Rende Township (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/212,715

(22) Filed: Sep. 18, 2008

(51) Int. Cl. A61F 9/02

(52) U.S. Cl. 2/428; 2/431; 2/452

(57) ABSTRACT

A protective goggle assembly is disclosed. Therein, a frame is provided with plural vent holes on an upper section and two lateral sides thereof More than one abutting portion is formed on an upper edge of the frame for abutting against an inner surface of a periphery of a lens when an engaging portion in a middle section of an upper end of the frame is engaged in a corresponding recess on the lens, thereby spacing the vent holes from the periphery of the lens. A buckle extended with a buckling section is bilaterally provided with protuberances which, after the buckle is assembled with the lens, must cross over corresponding upper and lower juts on the lens for re-entering two concave portions, so that the buckle can be disengaged. Thus, the lens is prevented from coming off the frame due to unintentional operation of the buckle.

### BACKGROUND OF THE INVENTION

1. Technical Field

The present invention relates to a protective goggle assembly, and more particularly, to a protective goggle assembly in which a frame coupled with an integrally formed lens has a periphery provided with a plurality of vent holes for introducing ambient air into an inside of a lens when the protective goggle assembly is in use, and in which buckles attached bilaterally to the lens and the frame are configured to prevent the lens from coming off the frame due to unintentional operation of the buckles.

2. Description of Related Art

Among the various goggle assemblies developed and designed by the related industry, an industrial protective goggle assembly for protecting the eyes from dust and powder is generally manufactured by fitting an integrally formed lens into a frame capable of conforming to the contour of a wearer's face, and then inserting buckles attached to two ends of a strap through two lateral sides of the frame and the lens so as to retain the frame and the lens in position. While this kind of protective goggle assemblies are in use, the buckles when touched by accident may be easily disengaged from corresponding portions of the lens and the frame, so that the lens also comes off the frame and thereby exposes the wearer's eyes to the risk of dust and powder pollution.

An example of such protective goggle assemblies is disclosed in U.S. Pat. No. 5,617,588 published on Apr. 8, 1997 and entitled “Snap Together Protective Goggle Construction with Toric Lens”, in which a protective goggle assembly comprises a rigid goggle body having a side adjacent to a wearer's face integrally formed with a resilient face-engaging portion. A tab and a detent are provided at an upper end and a lower end of a middle section of the goggle body, respectively, for engaging with a slot and a notch formed at corresponding locations on an integrally formed lens, respectively. Side edges extending bilaterally from the lens are correspondingly inserted into slots formed on two outer sides of the goggle body. A strap-receiving member is formed on each of two sides of the goggle body for receiving and retaining a strap passing therethrough. When the protective goggle assembly is worn by a user, the resilient face-engaging portion on an inner side of the goggle body is in contact with the user's face and cheekbones while the strap is wrapped around the user's head. Although this protective goggle assembly serves the intended function of eye protection, it is not comfortable to the face when worn by a user having a wider face or higher cheekbones, because the user's face may push the resilient face-engaging portion too hard toward the rigid goggle body.

### BRIEF SUMMARY OF THE INVENTION

The present invention provides an improvement over the shortcomings of existing industrial protective goggle assemblies during use, with the aim of proposing a protective goggle assembly in which a frame coupled with an integrally formed lens has a periphery provided with a plurality of vent holes for intruding ambient air into an inside of the lens while the protective goggle assembly is in use, and in which buckles coupled bilaterally to the lens and the frame can effectively prevent the lens from being disengaged from the frame due to unintentional operation of the buckles.

A primary objective of the present invention is to provide a protective goggle assembly wherein a frame is provided with a plurality of vent holes on an upper section and two lateral sides thereof and more than one abutting portion is formed at intervals on an upper edge of the frame formed with the plurality of vent holes for abutting stably against an inner surface of a periphery of an integrally formed lens when an engaging portion in a middle section of an upper end of the frame is engaged in a recess formed at a corresponding location on the lens during installation of the integrally formed lens, thereby spacing the periphery of the lens and each of the vent holes on the upper section and the two lateral sides of the frame, so as to facilitate introduction of ambient air though the vent holes into an inside of the lens Furthermore, a buckle is extended with a buckling section and bilaterally formed with protuberances which, after the buckle is assembled with the lens, must cross over corresponding upper and lower juts on the lens to re-enter two concave portions, so that the buckle can be disengaged. Thus, while the protective goggle assembly is in use, the lens is prevented from coming off the frame due to unintentional operation of the buckle.

### BRIEF DESCRIPTION OF THE SEVERAL VIEWS OF THE DRAWINGS

The invention as well as a preferred mode of use, further objectives and advantages thereof will be best understood by referring to the following detailed description of an illustrative embodiment in conjunction with the accompanying drawings, wherein:

FIG. 1 is a perspective view of a protective goggle assembly according to the present invention;

FIG. 2 is an exploded perspective view of the protective goggle assembly according to the present invention;

FIG. 3 is a sectional perspective view of the protective goggle assembly according to the present invention;

FIG. 4 is a partial front view of the protective goggle assembly according to the present invention;

FIG. 5 is a sectional view taken along a line 50-50 in FIG. 4;

FIG. 6 is a schematic drawing showing upward or downward rotation of a buckle of the protective goggle assembly according to the present invention;

FIG. 7A is a detailed drawing showing upward rotation of the buckle according to the present invention; and

FIG. 7B is a detailed drawing showing downward rotation of the buckle according to the present invention.

### DETAILED DESCRIPTION OF THE INVENTION

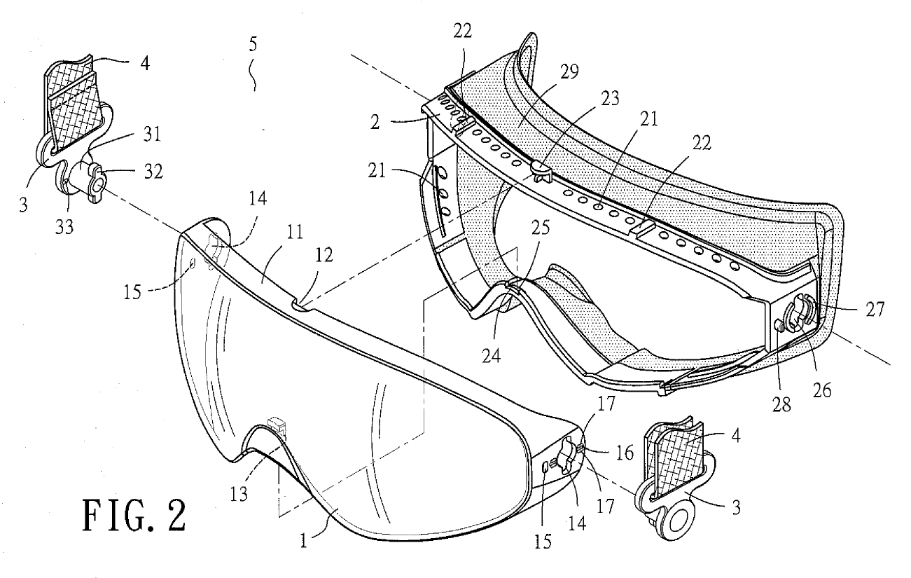

Referring to FIG. 2, a protective goggle assembly according to the present invention comprises an integrally formed lens 1, a frame 2 and a strap 4 having two ends each attached with a buckle 3.

The lens 1 is a rigid lens having an integrally formed periphery provided with a rearwardly extending covering edge 11. A recess 12 is formed in a middle section of an upper end of the lens 1 while a projection 13 extends from an inner surface of a nose-accommodating portion at a lower end of the lens 1. The lens 1 is bilaterally formed with particularly shaped through holes 14, each accompanied by an aperture 15 on one lateral side thereof. Besides, each of the through holes 14 is provided on each of two outer sides thereof with upper and lower juts 17 divided by a concave portion 16, as shown in FIG. 7A.

The frame 2 has a rear side integrally formed with a protective sheet 29 of a soft plastic, whereas other parts of the frame 2 are shaped according to the periphery of the lens 1. The frame 2 is provided with a plurality of vent holes 21 on an upper section and two lateral sides thereof. In addition, more than one abutting portion 22 is formed at intervals on an upper edge of the frame 2 formed with the plurality of vent holes 21. An engaging portion 23 is provided in a middle section of an upper end of the frame 2. A recessed section 24 is formed at a front edge of a middle section at a lower end of the frame 2, wherein an opening 25 is located adjacent to an inner surface of the recessed section 24. Moreover, the frame 2 is bilaterally provided with particularly shaped through holes 26, each of which is accompanied by a tenon 28 on a lateral side thereof and has a periphery formed with outwardly extending protrusions 27.

Each of the buckles 3 attached to the two ends of the strap 4 has one side extended with a buckling section 31 having an end formed with a boundary portion 32. In addition, each of the buckles 3 extended with the buckling sections 31 is bilaterally provided with protuberances 33 for being retained by the concave portions 16 on the outer sides of each of the particularly shaped through holes 14 provided bilaterally on the lens 1 during an earlier stage of assembling process.

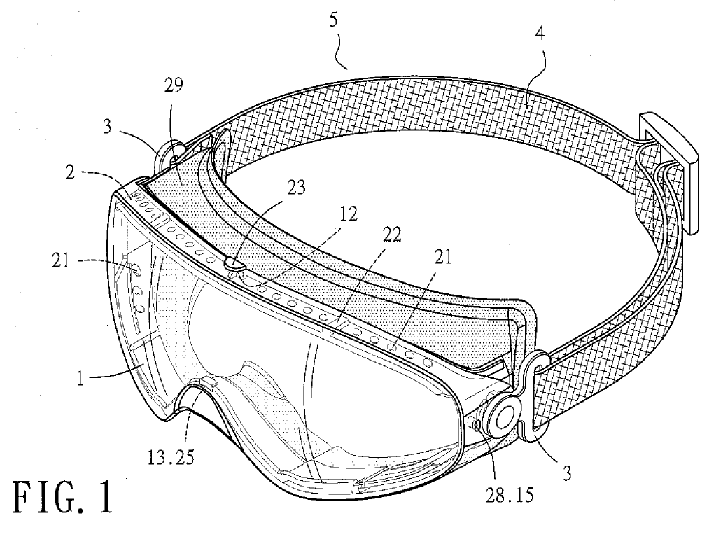

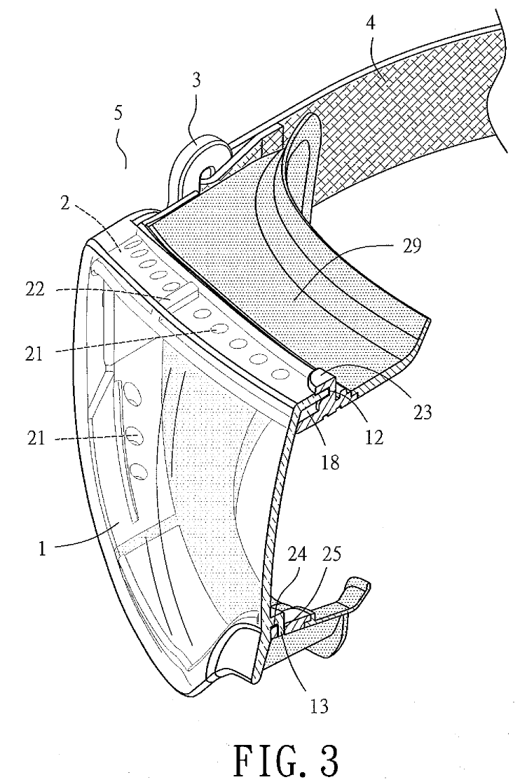

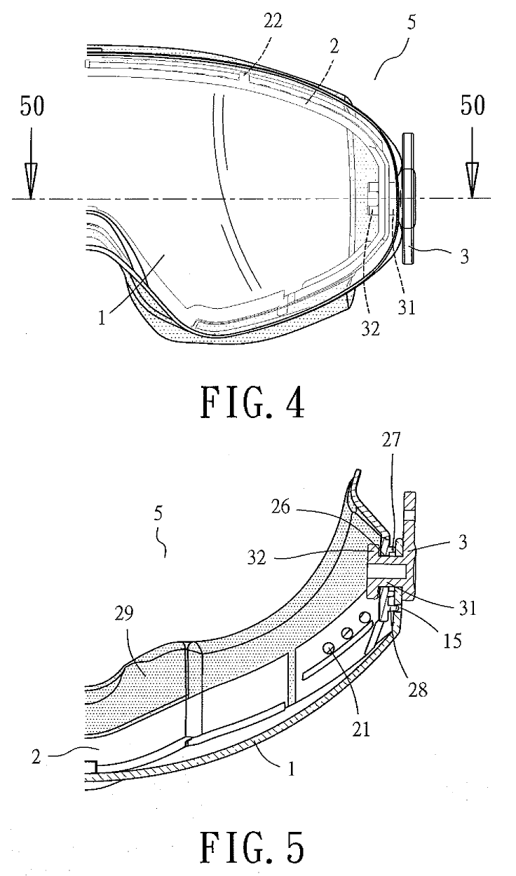

Referring to FIG. 3, in order to subsequently assemble the integrally formed lens 1, the frame 2 and the buckles 3 at the two ends of the strap 4, a front end of the frame 2 is correspondingly engaged with the covering edge 11 of the periphery of the lens 1, so that the engaging portion 23 at the upper end of the frame 2 is engaged in the recess 12 of the lens 1. Meanwhile, the opening 25 adjacent to the inner surface of the recessed section 24 at the front edge of the middle section of the lower end of the frame 2 is inserted by and thus retains the projection 13 at a corresponding end of the lens 1, also as shown in FIG. 3. Consequently, the abutting portions 22 formed at intervals on the upper edge of the frame 2 abut stably against an inner surface of the covering edge 11 of the lens 1, as shown in FIG. 3, so as to keep a space 18 between the covering edge 11 of the lens 1 and each of the vent holes 21 on the upper section and the two lateral sides of the frame 2, thereby facilitating introduction of ambient air through the vent holes 21 into an inside of the lens 1. Following that, the particularly shaped through holes 26 provided bilaterally on the frame 2 is aligned with the particularly shaped through holes 14 bilaterally provided on the lens 1, as shown in FIGS. 4 and 5, so that the protrusions 27 extending outward from the peripheries of the particularly shaped through holes 26 of the frame 2 press against an inner surface of the lens 1 while the tenons 28 on the one lateral sides of the particularly shaped through holes 26 of the frame 2 are inserted into the apertures. 15 at corresponding locations on the lens 1. Finally, the boundary portion 32 at the end of the buckling section 31 extending from the one side of each of the buckles 3 attached to the two ends of the strap 4 is inserted sequentially through the particularly shaped through holes 14 of the lens 1 and the particularly shaped through holes 26 of the frame 2, so that the protuberances 33 provided bilaterally near the buckling section 31 of each of the buckles 3 are disposed in the corresponding concave portions 16 of the lens 1 and thus retained by the adjacent upper and lower juts 17. Upon application of an external force to rotate the buckles 3 (by 90° in this illustrative embodiment), the protuberances 33 provided bilaterally on each of the buckles 3 are forced to cross over the adjacent upper and lower juts 17 of the lens 1, so that the boundary portion 32 at the end of the buckling section 31 of each of the buckles 3 presses stably against an inner surface of a corresponding one of the two particularly shaped through holes 26 of the frame 2, as shown in FIG. 5. Thus, the integrally formed lens 1, the frame 2 and the buckles 3 attached to the two ends of the strap 4 are easily assembled into a protective goggle assembly 5 ready for use, as shown in FIG. 1.

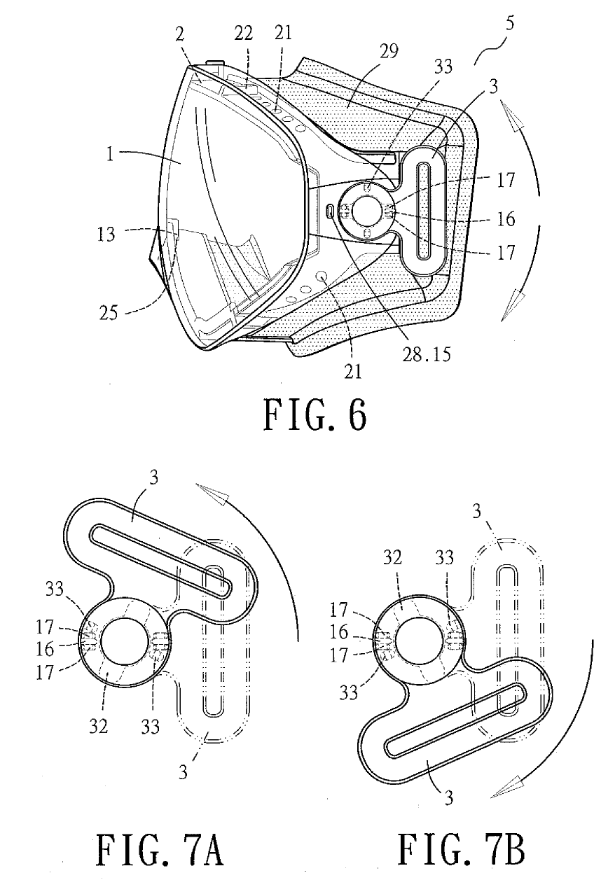

In order to prevent the lens 1 from coming off the frame 2 due to unintentional upper or downward rotation of the buckles 3 (as shown in FIG. 6) while the protective goggle assembly 5 is in use, the protuberances 33 formed on an inner side of each of the buckles 3 inserted through the lens 1 and the frame 2 are retained by the corresponding upper and lower juts 17 of the lens 1 (as shown in FIGS. 7A and 7B), so that the two protuberances 33 on the inner side of each of the buckles 3 are not allowed to enter the corresponding concave portions 16 of the lens 1. Hence, the lens 1 will not be disengaged from the frame 2 due to accidental operation of the buckles 3 while the protective goggle assembly 5 is in use.

In case a surface of the lens 1 of the protective goggle assembly 5 is worn and needs replacing, a user only has to rotate the buckles 3 by 90° (as shown in FIG. 7A or 7B) for forcing the protuberances 33 on the inner side of each of the buckles 3 to cross over the adjacent upper and lower juts 17 of the lens 1, so that the protuberances 33 on the one side of each of the buckles 3 are once again disposed in the concave portions 16 bilaterally provided near the particularly shaped through holes 14 of the lens 1. Thus, the buckles 3 inserted through the lens 1 and the frame 2 can be removed, allowing the lens 1 to be detached and replaced by a new lens.

The industrial protective goggle assembly described above provides the following advantages in use:

1. The vent holes on the upper section and the two lateral sides of the frame are spaced from the covering edge of the lens to facilitate introduction of ambient air through the vent holes into the inside of the lens. In consequence, good ventilation is achieved while dust and powder are prevented from entering the protective goggle assembly.

2. While in use, the protective goggle assembly can effectively prevent the lens from coming off the frame due to unintentional operation of the buckles.

1. A protective goggle assembly, comprising: an integrally formed lens; a frame shaped according to a periphery of the lens and having one side formed with a protective sheet of a soft material; and a strap having two ends each attached with a buckle; the protective goggle assembly being characterized in:
the lens being bilaterally formed with particularly shaped through holes, wherein each said through hole is provided on each of two outer sides thereof with upper and lower juts divided by a concave portion;
the frame being provided with a plurality of vent holes on an upper section and two lateral sides thereof, while more than one abutting portion is formed at intervals on an upper edge of the frame provided with the plurality of vent holes; and
each said buckle being extended with a buckling section provided bilaterally with protuberances;
wherein the protective goggle assembly is assembled by:
engaging an engaging portion in a middle section of an upper end of the frame into a recess provided at a corresponding location on the lens, so that the abutting portions formed at intervals on the upper edge of the frame abut stably against an inner surface of a covering edge of the lens, thereby spacing the covering edge of the lens from each said vent hole on the upper section and the two lateral sides of the frame, so as to facilitate introduction of ambient air through the vent holes into an inside of the lens;
inserting a boundary portion at an end of the buckling section extended from each said buckle sequentially through a corresponding one of the particularly shaped through holes of the lens and a corresponding one of particularly shaped through holes of the frame, so that the protuberances provided bilaterally near the buckling section of each said buckle are disposed in corresponding ones of the concave portions; and
forcing the protuberances to cross over adjacent said upper and lower juts of the lens, so that the boundary portion at the end of the buckling section of each said buckle presses stably against an inner surface of each said particularly shaped through hole of the frame;
whereby, after the buckles are assembled with the lens, the protuberances on each said buckle must cross over the adjacent upper and lower juts of the lens for re-entering the corresponding concave portions of the lens, so that the buckles can be disengaged, thereby preventing the lens from coming off the frame due to unintentional operation of the buckles while the protective goggle assembly is in use.
